(19) United States
(12) Patent Application Publication
(10) Pub. No.: US 2021/0207120 A1
(43) Pub. Date: Jul. 8, 2021

(54) CELL STRUCTURE BODY, PRODUCING METHOD FOR CELL STRUCTURE BODY, CELL CULTURING METHOD, AND MICRO FLOW PATH

(71) Applicant: FUJIFILM Corporation, Tokyo (JP)

(72) Inventors: Tomoaki KURAKAZU, Kanagawa (JP); Ryo MATSUNO, Kanagawa (JP); Kazunori TAKAHASHI, Kanagawa (JP)

(73) Assignee: FUJIFILM Corporation, Tokyo (JP)

(21) Appl. No.: 17/205,432

(22) Filed: Mar. 18, 2021

Related U.S. Application Data
(63) Continuation of application No. PCT/JP2019/030861, filed on Aug. 6, 2019.

(30) Foreign Application Priority Data
Sep. 28, 2018 (JP) 2018-185581

(51) Int. Cl. C12N 11/04; C12M 1/12; C12N 5/074; C12N 5/00

(52) U.S. Cl. CPC C12N 11/04; C12M 23/06; C12N 2533/74; C12N 5/0012; C12N 5/0696

(57) ABSTRACT

The producing method includes causing a cell suspension to flow into a tubular flow path; injecting a gel precursor into the flow path to cover the circumference of the cell suspension with the gel precursor; injecting a gas into the flow path to form, in the flow path, an alternating flow obtained from the gas and the cell suspension covered with the gel precursor; and injecting a gelating agent into the flow path and causing the gelating agent to join the alternating flow to the gel precursor.

### CROSS-REFERENCE TO RELATED APPLICATIONS

This application is a continuation application of International Application No. PCT/JP2019/030861, filed on Aug. 6, 2019, which is incorporated herein by reference in its entirety. Further, this application claims priority from Japanese Patent Application No. 2018-185581, filed on Sep. 28, 2018, the disclosure of which is incorporated by reference herein in their entirety.

### BACKGROUND

### Technical Field

The disclosed technique relates to a cell structure body, a producing method for a cell structure body, a cell culturing method, and a micro flow path.

### Related Art

As a technique relating to a cell structure body in which a circumference of a cell suspension containing cells is covered with a gel-like substance, for example, the following techniques are known.

For example, JP2016-539652A discloses a capsule containing a liquid core that contains cells having a hematopoietic function and a gel-like shell that surrounds the circumference of the liquid core. This capsule is formed by undergoing the following steps, a) a step of separately moving, in a jacket, a first liquid solution containing cells having a hematopoietic function and a second liquid solution containing a polyvalent liquid electrolyte capable of being gelated, b) a step of forming a series of small droplets at an outlet of the jacket so that each of the small droplets contains a central core which is formed of the first solution and a circumferential edge film which is formed of the second solution and completely covers the central core, c) a step of immersing each of the small droplets in a gel solution containing a reagent that can react with the polyvalent electrolyte of the film and causing the small droplets to be a gel state from a liquid state so that a gel-like shell is formed, thereby the central core forming a liquid core, and d) a step of collecting a formed capsule.

In addition, JP2017-154070A discloses a capsule encapsulating a biological substance, in which an inner film is a polymer having a communicating porous structure and a skin layer is present on an outer surface and an inner surface. Further, JP2017-154070A discloses a culture method for culturing a cell or a microorganism in a three-dimensional direction using this capsule.

Further, JP5633077B discloses a microfiber containing a micro gel fiber covered with a high-strength hydrogel and containing a cell or a cell culture in the micro gel fiber. Further, JP5633077B discloses that a microfiber consisting of two types of gels of an inner (core portion) gel and an outer (shell portion) gel is constructed by coaxially ejecting a sodium alginate solution before cross-linking separately into the core portion containing a cell and the shell portion, forming a fluid having a coaxial core-shell state, and introducing the fluid into an aqueous solution containing calcium chloride, thereby gelating the fluid.

In a case where stem cells such as an induced pluripotent stem cell (iPS cell) and an embryonic stem cell (ES cell) are cultured in a suspended state in a medium, the cells fuse with each other to form a cell aggregate (a sphere). However, in a case where the size of the cell aggregate (the sphere) is excessive, the supply of oxygen and nutrients to the central portion of the cell aggregate is insufficient, and the cells in the central portion of the aggregate may die by necrosis.

As a method for restricting the growth of the cell aggregate, a method in which a circumference of a cell suspension containing a plurality of cells is covered with a gel-like substance can be considered. As a producing method for a cell structure body in which a circumference of the cell suspension is covered with a gel-like substance, for example, there is a method for forming a cell structure body into a fibrous shape as disclosed in JP5633077B. However, the fibrous cell structure body is difficult to handle. For example, in a case where a plurality of fibrous cell structure bodies are dispersed in a medium and cultured, the fibrous cell structure bodies are entangled with each other to form a larger aggregate, whereby the supply of oxygen and nutrients to the cells may be insufficient.

As a result, for improving the handleability of the cell structure body, it is conceived that the fibrous cell structure body may be individualized. Examples of the conceivable method for individualization include a method in which a fibrous cell structure body is cut with a cutting tool such as a cutter after gelating of a gel-like substance and a method in which a pulling force is applied to a fibrous cell structure body to tear off the fibrous cell structure body.

However, in a case where the fibrous cell structure body is cut with a cutting tool, the exposed cut surface due to cutting is not covered with the gel-like substance, and thus a cell suspension may leak from the cut portion. In addition, in a case where the fibrous cell structure body is torn off to be individualized, the distal end of the torn-off part is stretched in the pulling direction, and thus the thickness of the gel-like substance in the torn-off part increases as compared with those of other parts. It may be difficult to uptake oxygen and nutrients from the part where the thickness of the gel-like substance is excessive. As described above, according to the method for individualizing the fibrous cell structure body after gelation of the gel-like substance, it is difficult to form a structure suitable for culturing.

As another producing method for a cell structure body in which a circumference of the cell suspension is covered with a gel-like substance, for example, there is a method for dropwise adding liquid droplets containing cells and a gel precursor into a gelating agent as disclosed in JP2016-539652A. However, according to this method, in a case where a liquid droplet containing cells and a gel precursor is dropwise added into a gelating agent, the liquid droplet is exposed to the atmosphere, and there is a risk that the cells are contaminated with a contamination source such as a bacterium or a virus.

In addition, the shape of the cell structure body produced by this method is spherical. In a case where the shape of the cell structure body is spherical, the ratio of the volume of the gel-like substance to the volume of the cells increases. Here, it is assumed that the gel-like substance that covers the cells is removed in the subsequent step. It is assumed that the gel-like substance is removed by dissociating the gel-like substance with a chelating agent such as ethylenediaminetetraacetic acid (EDTA). In a case where the ratio of the volume of the gel-like substance to the volume of the cells is large, the time for treating a chelating agent becomes long, and thus the damage to the cells becomes large.

### SUMMARY

The disclosed technique provides, for a cell structure body in which a circumference of a cell suspension containing a plurality of cells is covered with a gel-like substance, a cell structure body having a structure suitable for culturing, a producing method for a cell structure body, a cell culturing method, and a micro flow path.

A producing method for a cell structure body according to the disclosed technique is a producing method for a cell structure body in which a circumference of a cell suspension containing a plurality of cells is covered with a gel-like substance, and the producing method includes a cell suspension flowing step of causing a cell suspension to flow into a tubular flow path; a covering step of injecting a gel precursor into the flow path to cover the circumference of the cell suspension with the gel precursor; an alternating flow forming step of injecting a gas into the flow path to form, in the flow path, an alternating flow obtained from the gas and the cell suspension covered with the gel precursor; and a gelating step of injecting a gelating agent into the flow path and causing the gelating agent to join the alternating flow to gelate the gel precursor. According to the producing method for a cell structure body according to the disclosed technique, for a cell structure body in which a circumference of a cell suspension containing a plurality of cells is covered with a gel-like substance, a structure suitable for culturing can be formed, and the individualization of the cell suspension covered with a gel precursor can be suitably performed.

In the alternating flow forming step, an injection amount of the gas that is injected into the flow path per unit time may be constant. This makes it possible to stably form an alternating flow obtained from the gas and the cell suspension covered with the gel precursor.

In the alternating flow forming step, an aspect ratio of the cell structure body may be controlled by an injection amount of the gas that is injected into the flow path per unit time. This makes it possible to easily control the aspect ratio of the cell structure body.

In addition, in the alternating flow forming step, the gas may be intermittently injected into the flow path. Further, a cross section of the flow path, which is orthogonal to a liquid passage direction, may have a circular or polygonal shape.

A contact angle of the gel precursor with respect to an inner wall surface of the flow path is preferably 50° or more. In addition, the inner wall surface of the flow path is preferably subjected to a water repellency treatment. This makes it possible to stably form an alternating flow obtained from the gas and the cell suspension covered with the gel precursor, and the individualization of the cell suspension covered with a gel precursor can be suitably performed.

A cell culturing method according to the disclosed technique is a cell culturing method for culturing cells contained in a cell structure body produced by the producing method described above and includes a culturing step of culturing the cell structure body in a medium. According to the cell culturing method according to the disclosed technique, the aggregation of the cell structure bodies in cell culture can be avoided with damage to cells in the gel-like substance removal step being reduced.

The cell culturing method according to the disclosed technique may further include a removing step of removing the gel-like substance after the culturing step.

A cell structure body according to the disclosed technique includes a cell suspension containing a plurality of cells and a gel-like substance covering an entire circumference of the cell suspension. In the cell structure body, a thickness of the gel-like substance is 0.1 times or more and 3 times or less of an average thickness of the gel-like substance, over an entire region, and the cell structure body has a non-spherical outer shape and an aspect ratio of 100 or less. According to the cell structure body according to the disclosed technique, a cell structure body suitable for culturing is provided.

In the cell structure body according to the disclosed technique, the aspect ratio is preferably 20 or less. This makes it possible to further enhance the handleability of the cell structure body.

In the cell structure body according to the disclosed technique, a length in a lateral direction is preferably is 10 μm or more and 500 μm or less. In a case where the length of the cell structure body in the lateral direction is set to be 10 μm or more, it is possible to suitably inject the cell suspension into the micro flow path. In addition, in a case where the length of the cell structure body in the lateral direction is set to be 500 μm or less, it is possible to supply a sufficient amount of oxygen and nutrients to the central portion of the core portion in a case where the cell structure body is dispersed in a medium and cultured.

A micro flow path according to the disclosed technique includes a tubular main flow path through which a cell suspension flows; a first branch flow path that communicates with a first injection port into which a gel precursor is injected, and joins the main flow path at a first joining portion; a second branch flow path that communicates with a second injection port into which a gas is injected, and joins the main flow path at a second joining portion of the main flow path, the second joining portion being disposed downstream of the first joining portion in a liquid passage direction; and a third branch flow path that communicates with a third injection port into which a gelating agent gelating the gel precursor is injected, and joins the main flow path at a third joining portion of the main flow path, the third joining portion being disposed downstream of the second joining portion in the liquid passage direction. According to the micro flow path according to the disclosed technique, a cell structure body suitable for culturing can be easily formed.

According to the disclosed technique, as one aspect, a structure suitable for culturing can be formed for a cell structure body in which a circumference of a cell suspension containing a plurality of cells is covered with a gel-like substance.

### BRIEF DESCRIPTION OF THE DRAWINGS

FIG. 1 is a view schematically illustrating a structure of a cell structure body according to an embodiment of the disclosed technique.

FIG. 2 is a view schematically illustrating a structure of a cell structure body according to Comparative Example 3.

FIG. 3 is a view for explaining a measuring method for an average thickness of a shell portion in the cell structure body according to the embodiment of the disclosed technique.

FIG. 4 is a cross-sectional view illustrating one example of a configuration of a micro flow path that is used for producing the cell structure body according to the embodiment of the disclosed technique.

FIG. 5 is a step flow chart illustrating one example of a producing method for a cell structure body according to the embodiment of the disclosed technique.

FIG. 6 is a view illustrating a state in the micro flow path in a case where the cell structure body according to the embodiment of the disclosed technique is produced.

FIG. 7 is a view illustrating a measuring method for a contact angle of a gel precursor with respect to an inner wall of the micro flow path according to the embodiment of the disclosed technique.

FIG. 8A is a phase-contrast microscope image of a cell structure body produced by the producing method according to the embodiment of the disclosed technique.

FIG. 8B is a schematic view of a cell structure body corresponding to FIG. 8A.

FIG. 9A is a phase-contrast microscope image of a cell structure body according to Comparative Example 1.

FIG. 9B is a schematic view of a cell structure body corresponding to FIG. 9A.

FIG. 10A is a phase-contrast microscope image of a cell structure body according to Comparative Example 2.

FIG. 10B is a schematic view of a cell structure body corresponding to FIG. 10A.

FIG. 11 is a step flow chart illustrating one example of a cell culturing method according to the embodiment of the disclosed technique.

FIG. 12 is a view illustrating one example of the cell culturing method according to the embodiment of the disclosed technique.

### DETAILED DESCRIPTION

Hereinafter, one example of the embodiment of the disclosed technique will be described with reference to the drawings. In each drawing, the same or equivalent configuration elements or parts are designated by the same reference numeral.

### First Embodiment

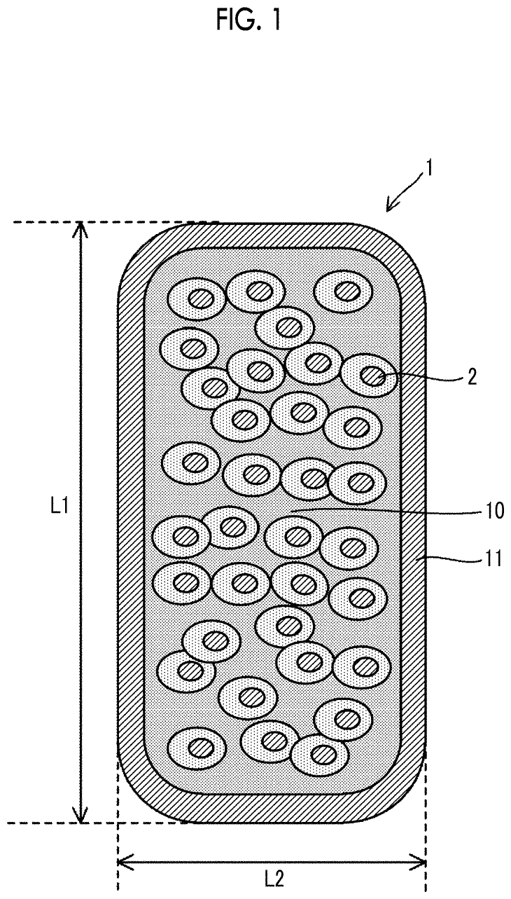

FIG. 1 is a view schematically illustrating a structure of a cell structure body 1 according to the embodiment of the disclosed technique. The cell structure body 1 includes a core portion 10 consisting of a cell suspension containing a plurality of cells 2, and a shell portion 11 consisting of a gel-like substance covering the entire circumference of the core portion 10.

The kind of the cell 2 constituting the core portion 10 is not particularly limited, but for example, stem cells such as an iPS cell and an ES cell can be used. The cell suspension constituting the core portion 10 contains a medium that provides a growth environment for the cell 2.

The gel-like substance constituting the shell portion 11 is preferably a hydrogel having dissociability. “Having dissociability” means having a property of being dissolved by treatment with a chelating agent such as EDTA. As the hydrogel having dissociability, for example, a gelated sodium alginate can be used.

The outer shape of the cell structure body 1 may be non-spherical and may be, for example, cylindrical, quadrangular columnar, or hexagonal columnar. In addition, the aspect ratio of the cell structure body 1 is preferably more than 1 and 100 or less and more preferably 1.1 or more and 20 or less. The aspect ratio is the ratio (L1/L2) of the length L1 in the longest part to the length L2 in the shortest part of the outer shape length of the cell structure body 1.

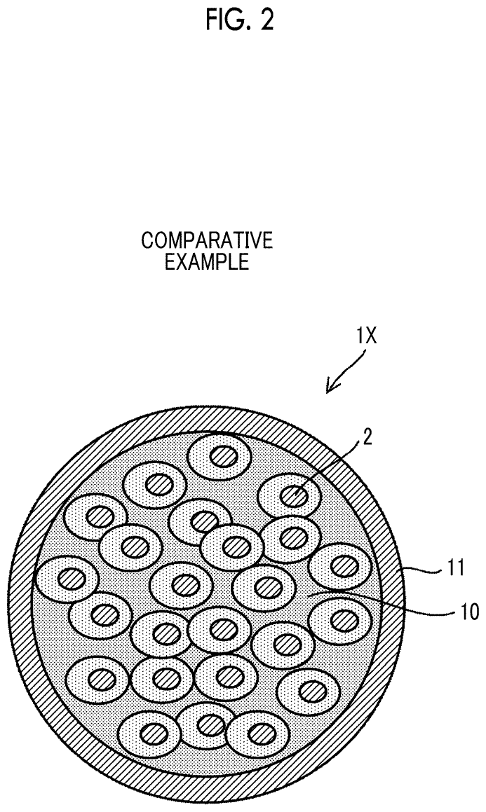

FIG. 2 is a view schematically illustrating a structure of a cell structure body 1X according to Comparative Example 3. The shape of the cell structure body 1X according to Comparative Example 3 is spherical. That is, the aspect ratio of the cell structure body 1X according to Comparative Example 3 is 1. The ratio (hereinafter, referred to as a gel volume fraction) of the volume of the shell portion 11 (gel-like substance) to the volume of the core portion 10 (cell suspension) in the cell structure body 1X according to Comparative Example 3 having a spherical outer shape is greater than that in the cell structure body 1 (see FIG. 1) according to the present embodiment having a non-spherical outer shape. That is, the closer the aspect ratio of the cell structure body is to 1, the greater the gel volume fraction is.

In a case of assuming that the diameter of the cell structure body 1X according to Comparative Example 3 is, for example, 200 μm, and the thickness of the gel-like substance constituting the shell portion 11 is, for example, 50 μm, the gel volume fraction is about 8. In addition, in a case of assuming that the outer shape of the cell structure body 1 according to the present embodiment is, for example, a cylindrical shape, the length L1 in the longitudinal direction is, for example, 2 mm, the length L2 in the lateral direction is, for example, 200 μm, and the thickness of the gel-like substance constituting the shell portion 11 is, for example, 50 μm, the gel volume fraction is about 4.2.

It is assumed that the gel-like substance constituting the shell portion 11 is removed in the subsequent step. The removal of the gel-like substance is performed by dissociating the gel-like substance with a chelating agent such as EDTA. In a case where the gel volume fraction is greater, the time for treating a chelating agent becomes long, and the damage to the cell 2 becomes large. Since the outer shape of the cell structure body 1 according to the present embodiment is non-spherical and the aspect ratio is greater than 1, the gel volume fraction can be reduced as compared with that of the cell structure body 1X according to Comparative Example 3 having a spherical outer shape, and the time for treating a chelating agent when removing the gel-like substance constituting the shell portion 11 can be shortened. In particular, in a case where the outer shape of the cell structure body 1 is to be cylindrical, the gel volume fraction can be reduced. Further, in a case where the outer shape of the cell structure body 1 is to be a quadrangular columnar shape or a hexagonal columnar shape, although the gel volume fraction is increased as compared with the case where the cell structure body 1 has a cylindrical shape having the same size as the quadrangular columnar shape or the hexagonal columnar shape, the surface area of the cell structure body 1 can be increased, which is advantageous in terms of the uptake of oxygen and nutrients in the core portion 10.

Further, since the cell structure body 1 according to the present embodiment has an aspect ratio of 100 or less, for example, the handleability of the cell structure body 1 in the culturing step of culturing the cell structure body 1 can be improved. That is, in a case where a plurality of the cell structure bodies 1 are dispersed in a medium and cultured, the risk of entanglement of the cell structure bodies can be reduced. In a case where the aspect ratio is set to be 20 or less, the handleability of the cell structure body 1 can be further improved.

In addition, the length L2 of the cell structure body 1 according to the present embodiment in the lateral direction is preferably 10 μm or more and 500 μm or less. In a case where the length L2 of the cell structure body 1 in the lateral direction is set to be 10 μm or more, it is possible to suitably inject the cell suspension into the micro flow path described later that is used for producing the cell structure body 1. In addition, in a case where the length L2 of the cell structure body 1 in the lateral direction is set to be 500 μm or less, it is possible to supply a sufficient amount of oxygen and nutrients to the central portion of the core portion 10 in a case where the cell structure body 1 is dispersed in a medium and cultured.

In the cell structure body 1 according to the present embodiment, the gel-like substance constituting the shell portion 11 covers the entire circumference of the cell suspension constituting the core portion 10 without any defect. That is, the shell portion 11 covers the core portion 10 with a sufficient thickness even at the end portion of the cell structure body 1 in the longitudinal direction. Further, the thickness of the shell portion 11 is 0.1 times or more and 3 times or less the average thickness of the shell portion 11 over the entire region of the shell portion 11. The average thickness of the shell portion 11 is determined as follows.

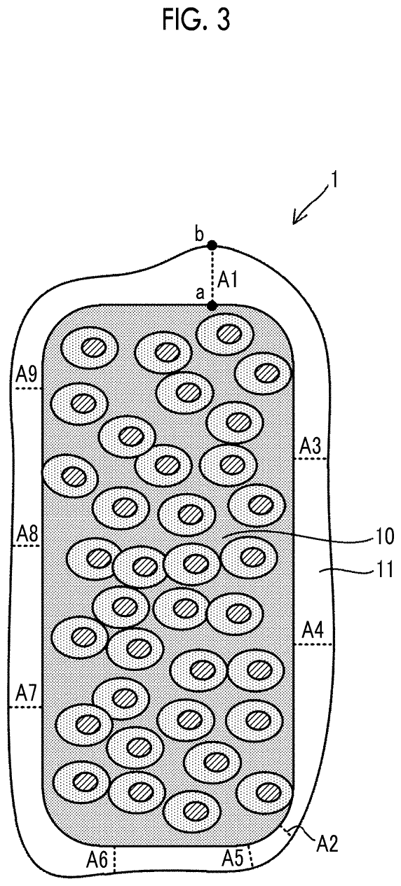

That is, ten cell structure bodies 1 are randomly extracted, and each of the extracted cell structure bodies 1 is observed under a phase-contrast microscope. For each of the cell structure bodies 1, as illustrated in FIG. 3, a total of nine parts A1 to A9 including a part A1 at which the thickness of the gel-like substance constituting the core portion 10 is largest, a part A2 where the thickness thereof is smallest, and starting from the thickest part A1, parts A3 to A9 that divide the shell portion 11 into eight equal parts are extracted, and an average value Xi obtained by averaging the thicknesses of these nine parts A1 to A9 is determined. The average value X of the respective average values Xi determined for the ten cell structure bodies 1 is defined as the average thickness of the shell portion 11. In each of the parts A1 to A9 of the shell portion 11, the thickness means a length of a line segment ab corresponding to each of the parts, where the line segment ab connects a point a of the inner wall of the shell portion 11 and a point b at which the line perpendicularly extended from the point a to the inner wall intersects the outer wall of the shell portion 11.

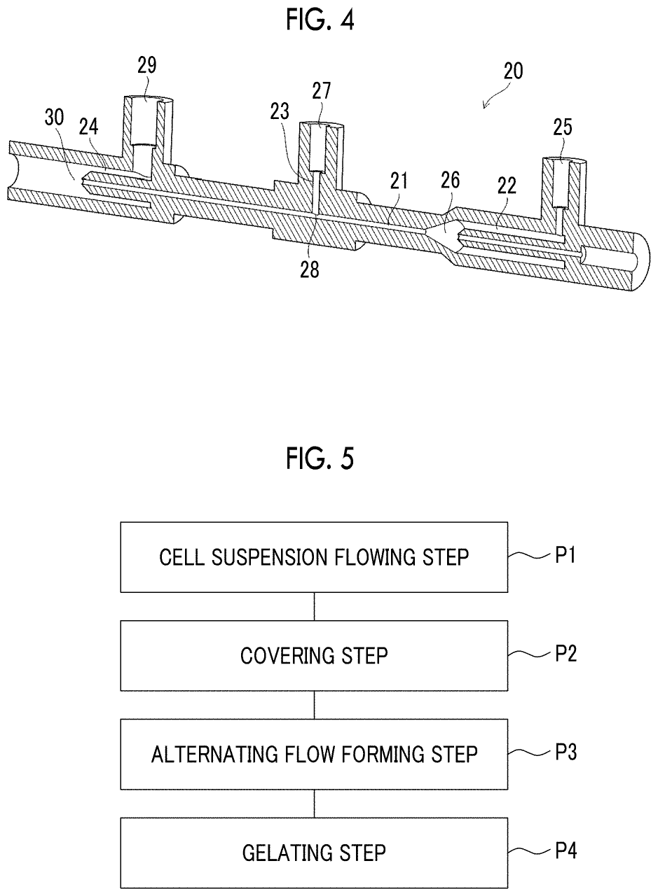

The producing method for the cell structure body 1 will be described below. FIG. 4 is a cross-sectional view illustrating one example of a configuration of a micro flow path 20 that is used for producing the cell structure body 1. The micro flow path 20 is a device having a flow path formed by using a microfabrication technique such as micro electro mechanical systems (MEMS) technology. The material of the micro flow path 20 is not particularly limited, but glass, polydimethylsiloxane (PDMS), quartz, a resin, and the like can be used. The micro flow path 20 includes a main flow path 21, a first branch flow path 22, a second branch flow path 23, and a third branch flow path 24.

The main flow path 21 is a tubular flow path through which the cell suspension constituting the core portion 10 of the cell structure body 1 flows. In the present embodiment, the main flow path 21 extends in a straight line, and the flow direction of the cell suspension is linear in the micro flow path 20. The shape of the cross section of the main flow path 21 orthogonal to the liquid passage direction (the flow direction of the cell suspension) is not particularly limited, but the cross section may have, for example, a circular or elliptical shape, or a polygonal shape such as quadrangular shape or hexagonal shape. The tube diameter of the main flow path 21 corresponds to the length L2 of the cell structure body 1 to be produced, in the lateral direction. Accordingly, the tube diameter of the main flow path 21 is set according to the target size of the cell structure body 1 to be produced.

The first branch flow path 22 communicates with a first injection port 25 into which a gel precursor, which is a material for the gel-like substance constituting the shell portion 11 of the cell structure body 1, is injected. The first branch flow path 22 joins the main flow path 21 at a first joining portion 26 on the main flow path 21.

The second branch flow path 23 communicates with a second injection port 27 into which a gas is injected. The second branch flow path 23 joins the main flow path 21 at a second joining portion 28 of the main flow path 21, where the second joining portion 28 is disposed downstream of the first joining portion 26 in a liquid passage direction (a flow direction of the cell suspension).

The third branch flow path 24 communicates with a third injection port 29 into which a gelating agent gelating the gel precursor is injected. The third branch flow path 24 joins the main flow path 21 at a third joining portion 30 of the main flow path 21, where the third joining portion 30 is disposed downstream of the second joining portion 28 in the liquid passage direction (the flow direction of the cell suspension).

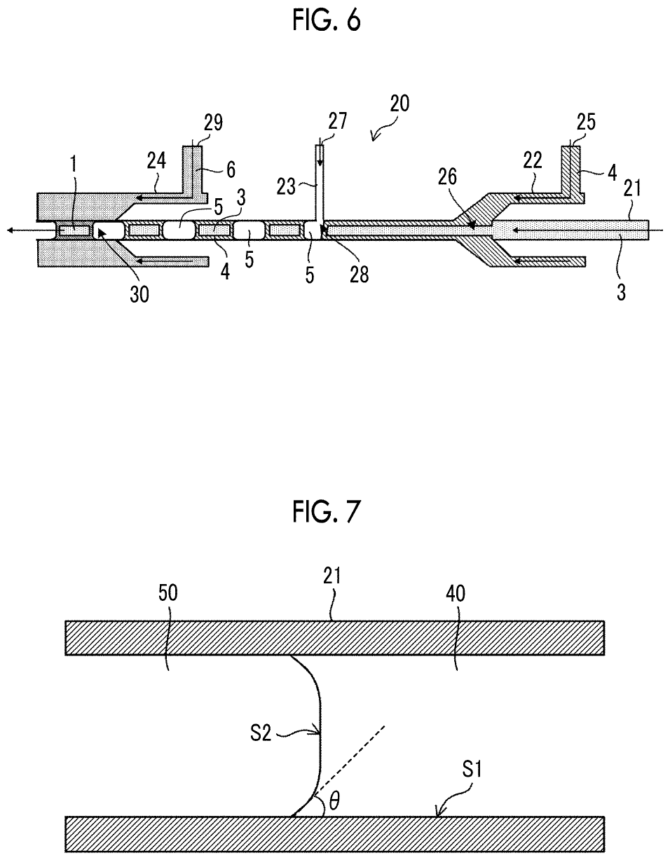

FIG. 5 is a step flow chart illustrating one example of a producing method for a cell structure body 1. The producing method for the cell structure body 1 includes a cell suspension flowing step P1, a covering step P2, an alternating flow forming step P3, and a gelating step P4. FIG. 6 is a view illustrating a state in the micro flow path 20 In a case where the cell structure body 1 is produced.

In the cell suspension flowing step P1, the cell suspension 3 containing a plurality of cells is injected into the main flow path 21, and the cell suspension 3 is caused to flow into the main flow path 21. The injection of the cell suspension 3 is performed, for example, using a syringe. It is preferable to suppress the precipitation of cells, for example, by stirring the cell suspension 3 in the syringe so that the density of cells contained in the cell suspension 3 is uniform in the micro flow path 20. In addition, it is preferable to keep the flow rate of the cell suspension 3 flowing in the main flow path 21 constant by keeping the injection amount of the cell suspension 3 that is injected into the main flow path 21 per unit time constant. The injected cell suspension 3 flows downstream along the main flow path 21.

In the covering step P2, a gel precursor 4 is injected from the first injection port 25, and the circumference of the cell suspension 3 flowing in the main flow path 21 is covered with the gel precursor 4. The gel precursor 4 injected from the first injection port 25 joins the cell suspension 3 flowing in the main flow path 21 at the first joining portion 26 via the first branch flow path 22, thereby covering the circumference of the cell suspension 3. The gel precursor 4 is a material for the gel-like substance constituting the shell portion 11 of the cell structure body 1. As the gel precursor 4, for example, an aqueous solution of sodium alginate can be used. It is preferable to keep the injection amount of the gel precursor 4 that is injected into the first branch flow path 22 per unit time constant and keep the flow rate of the gel precursor 4 flowing in the first branch flow path 22 constant. The cell suspension 3 covered with the gel precursor 4 flows further downstream along the main flow path 21.

In the alternating flow forming step P3, a gas 5 is injected from the second injection port 27 to form an alternating flow obtained from the gas 5 and the cell suspension 3 covered with the gel precursor 4 in the main flow path 21. The gas 5 injected from the second injection port 27 joins the cell suspension 3 covered with the gel precursor 4 flowing in the main flow path 21 at the second joining portion 28 via the second branch flow path 23. The gas 5 comes into contact with the cell suspension 3 covered with the gel precursor 4, thereby forming, in the main flow path 21, an alternating flow that flows in a state where the cell suspension 3 covered with the gel precursor 4 and the gas 5 are alternately disposed. That is, the cell suspension 3 covered with the gel precursor 4 is fragmented and individualized by the gas 5. The gel precursor 4 wraps around the surface of the individualized cell suspension, on the side of the surface in contact with the gas 5. As a result, the entire circumference of the individualized cell suspension is covered with the gel precursor 4.

In the alternating flow forming step P3, it is preferable that the injection amount of the gas 5 that is injected from the second injection port 27 per unit time is constant. In a case where the injection amount of the gas 5 per unit time is set to be constant, the length of the cell suspension 3 covered with the gel precursor 4 in the liquid passage direction and the length of the gas 5 in the liquid passage direction in the alternating flow can be kept constant. That is, the length of the individual pieces of the cell suspension 3 covered with the gel precursor 4 can be kept constant, whereby the length L1, in the longitudinal direction, of the cell structure body 1 which is produced by the present producing method can be kept constant. In addition, “keeping the injection amount of the gas 5 per unit time constant” means that the length L1, in the longitudinal direction, of the cell structure body 1 which is produced by the present producing method may be within the variation that is allowed depending on the purpose of using the cell structure body 1 and does not mean keeping constant in the strict sense.

The injection of the gas 5 into the micro flow path 20 may be continuous or intermittent. In a case where the gas 5 is injected intermittently, a commercially available dispenser may be used, or the opening degree of the mass flow controller may be adjusted to an intermittent pattern. Further, for preventing the cell suspension 3 covered with the gel precursor 4 from entering the second branch flow path 23, the pressure at the time of injecting the gas 5 is preferably 0.1 MPa or more. In addition, the tube diameter of the second branch flow path 23 through which the gas 5 passes is preferably 0.1 mm or more and 0.5 mm or less and preferably as thin as possible within the above range.

As the gas 5, a gas having no cytotoxicity, for example, air, oxygen, nitrogen, and carbon dioxide can be used. Further, the gas 5 is preferably adjusted to have a composition and temperature suitable for culturing the cells contained in the cell suspension 3. As the gas 5, for example, a gas containing 5% carbon dioxide, 20% oxygen, and 75% nitrogen can be preferably used, and it is more preferable for the temperature of the gas to be adjusted to 37° C.

Here, for stably forming the alternating flow obtained from the gas 5 and the cell suspension 3 covered with the gel precursor 4, the inner wall of the main flow path 21 preferably has relatively high water repellency to the gel precursor 4. In a case where the water repellency of the inner wall of the main flow path 21 to the gel precursor 4 is insufficient, the gel precursor 4 wraps around the space between the gas 5 and the inner wall of the main flow path 21, and the circumference of the gas 5 may be covered with the gel precursor 4. That is, in a case where the water repellency of the inner wall of the main flow path 21 to the gel precursor 4 is insufficient, the gas 5 becomes bubbles in the main flow path 21, and thus it is difficult to stably form the alternating flow obtained from the gas 5 and the cell suspension 3 covered with the gel precursor 4. In this case, it is difficult to properly individualize the cell suspension 3 covered with the gel precursor 4.

Examples of the method for increasing the water repellency of the inner wall of the main flow path 21 to the gel precursor 4 include a method for impregnating the inner wall of the main flow path 21 with a silane coupling agent having a hydrophobic group such as a perfluoroalkyl chain. Examples of another method include a method for performing irradiation with CF-based plasma from the end portion of the main flow path 21. In a case where the hydrophobicity of the material of the inner wall of the main flow path 21 and the treatment conditions are adjusted, the desired water repellency can be obtained.

The water repellency of the inner wall of the main flow path 21 to the gel precursor 4 can be quantified by the contact angle θ of the gel precursor 4 with respect to the inner wall of the main flow path 21. In the present specification, the contact angle θ means a contact angle measured by the following measuring method. FIG. 7 is a view illustrating a measuring method for the contact angle θ. As illustrated in FIG. 7, a predetermined amount of the gel precursor 40 is injected into the main flow path 21. The angle which is formed by an interface S2 of the gas 50 and the gel precursor 40, where interface S2 is formed in the main flow path 21, and the inner wall surface S1 of the main flow path 21 is defined as the contact angle θ.

The contact angle θ of the gel precursor 4 with respect to the inner wall of the main flow path 21 is preferably 50° or more. In the alternating flow forming step P3, in a case where the contact angle θ is set to be 50° or more, it is possible to stably form the alternating flow obtained from the gas 5 and the cell suspension 3 covered with the gel precursor 4, and the individualization of the cell suspension 3 covered with the gel precursor 4 can be suitably performed.

In the gelating step P4, a gelating agent 6 is injected from the third injection port 29, and the gelating agent 6 is caused to join the alternating flow obtained from the gas 5 and the cell suspension 3 covered with the gel precursor 4, thereby gelating the gel precursor 4. The gelating agent 6 injected from the third injection port 29 joins the alternating flow flowing in the main flow path 21 at the third joining portion 30 via the third branch flow path 24. In a case where the gelating agent 6 comes into contact with the gel precursor 4 that covers the cell suspension 3, a cross-linking reaction occurs in the gel precursor 4, and the gel precursor 4 is gelated. As a result, the shell portion 11 consisting of a gel-like substance is formed. Since the gel precursor 4 is gelated while maintaining the state of covering the entire circumference of the cell suspension 3, the entire circumference of the cell suspension 3 is covered with the gel-like substance without any defect. As the gelating agent 6, for example, an aqueous solution of calcium chloride can be used. Individual pieces of the cell structure body 1 in which the circumference of the cell suspension 3 is covered with a gel-like substance are continuously discharged from the end portion downstream of the micro flow path 20.

As described above, according to the producing method for the cell structure body 1 according to the embodiment of the disclosed technique, in the alternating flow forming step P3, since the alternating flow obtained from the gas 5 and the cell suspension 3 covered with the gel precursor 4 is formed in the main flow path 21, the cell suspension 3 covered with the gel precursor 4 is fragmented and individualized. Thereafter, in the gelating step P4, the gel precursor 4 is gelated while maintaining the state of covering the entire circumference of the cell suspension 3. That is, according to the producing method according to the present embodiment, since the individualization of the cell structure body 1 is substantially completed in the alternating flow forming step P3, it is not necessary to cut or tear off the cell structure body 1 after gelation.

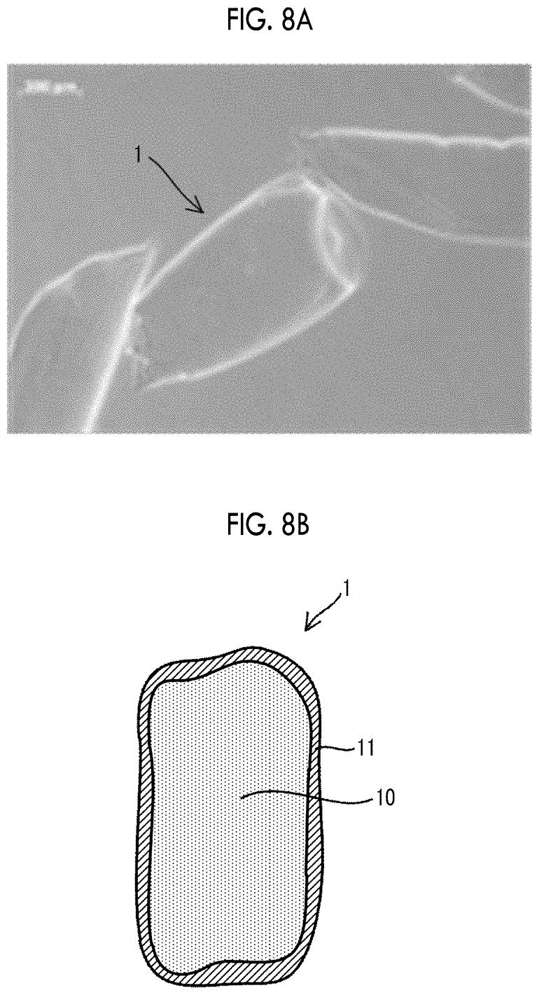

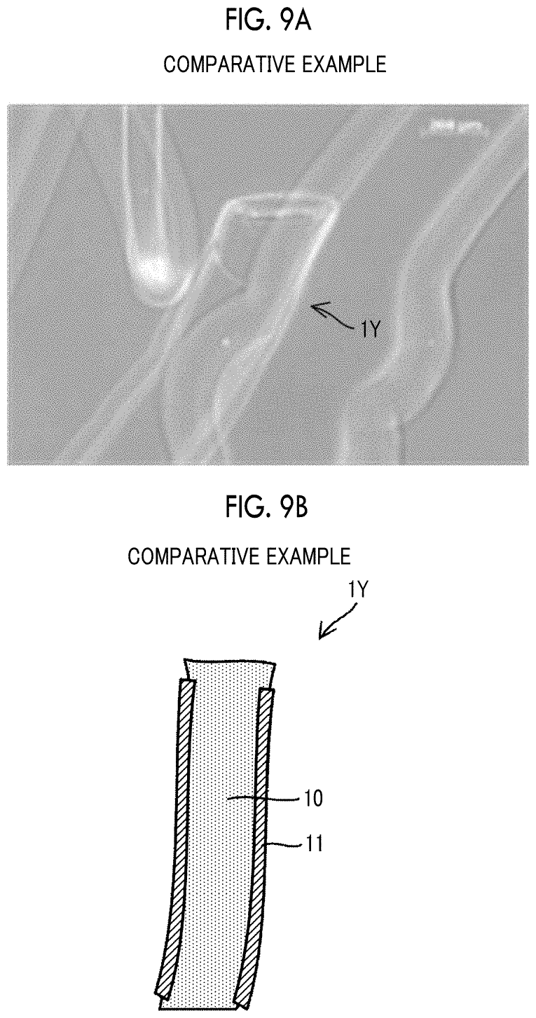

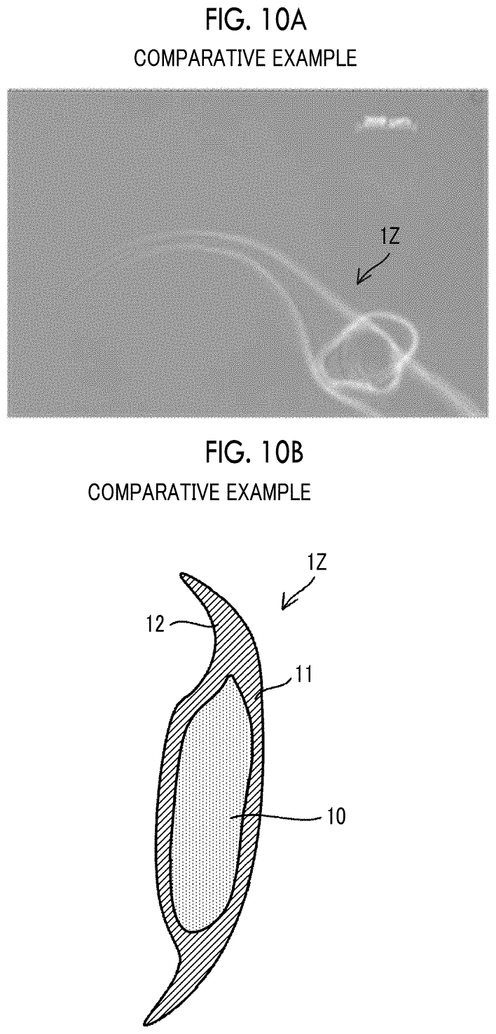

FIG. 8A is a phase-contrast microscope image of a cell structure body 1 produced by the producing method according to the embodiment of the disclosed technique, and FIG. 8B is a schematic view of the cell structure body 1, corresponding to FIG. 8A. FIG. 9A is a phase-contrast microscope image of a cell structure body 1Y according to Comparative Example 1, in which a fibrous cell structure body is individualized by cutting after the gelation of the shell portion, and FIG. 9B is a schematic view of the cell structure body 1Y, corresponding to FIG. 9A. FIG. 10A is a phase-contrast microscope image of a cell structure body 1Z according to Comparative Example 2, in which a fibrous cell structure body is individualized by tearing off after the gelation of the shell portion, and FIG. 10B is a schematic view of the cell structure body 1Z, corresponding to FIG. 10A.

According to the cell structure body 1Y according to Comparative Example 1, since the cut surface is not covered with the shell portion 11 and the core portion 10 is exposed from the cut surface as illustrated in FIG. 9B, the cell suspension of the core portion 10 leaks from the cut surface. On the other hand, according to the producing method for a cell structure body 1 according to the embodiment of the disclosed technique, it is not necessary to cut the cell structure body after gelation of the shell portion 11, and thus the leakage of the cell suspension due to individualization can be avoided.

According to the cell structure body 1Z according to Comparative Example 2, as illustrated in FIG. 10B, the distal end of a torn-off part 12 is stretched in the pulling direction, and thus the thickness of the shell portion 11 in the torn-off part 12 is great as compared with those of the other parts. It may be difficult to uptake oxygen and nutrients from the part where the thickness of the shell portion 11 is excessive. On the other hand, according to the producing method for a cell structure body 1 according to the embodiment of the disclosed technique, it is not necessary to tear off the cell structure body after gelation of the shell portion 11, and thus the variation of the thickness of the shell portion 11 can be suppressed.

In addition, according to the producing method according to the embodiment of the disclosed technique, the gel precursor 4 wraps around the surface of the cell suspension 3 individualized in the alternating flow forming step P3, on the side of the surface in contact with the gas 5, and thus the entire circumference of the individualized cell suspension 3 is covered with the gel precursor 4. Thereafter, in the gelating step P4, the gel precursor 4 is gelated while maintaining the state of covering the entire circumference of the cell suspension 3. As a result, the occurrence of defects in the shell portion 11 can be suppressed, and the thickness variation in each part of the shell portion 11 can be suppressed.

In addition, according to the producing method according to the embodiment of the disclosed technique, it is possible to adjust the lengths of the individual pieces of the cell suspension 3 covered with the gel precursor in the liquid passage direction (the flow direction of the cell suspension) by adjusting the ratio of the injection amount of the cell suspension 3 that is injected into the micro flow path 20 per unit time to the injection amount of the gas 5 that is injected into the micro flow path 20 per unit time. As a result, it is possible to adjust the aspect ratio of the cell structure body 1 to be produced. For example, in a case where the injection ratio of the gas 5 to the cell suspension 3 is increased, the length L of the cell structure body 1 in the longitudinal direction can be shortened, and thus the aspect ratio can be reduced. In addition, the aspect ratio of the cell structure body 1 to be produced can also be adjusted by setting the tube diameter of the main flow path 21. For example, in a case where the tube diameter of the main flow path 21 and the injection amount of the cell suspension 3 that is injected into the micro flow path 20 per unit time are fixed, it is possible to adjust the aspect ratio of the cell structure body 1 to be produced, by adjusting the injection amount of the gas 5 that is injected into the micro flow path 20 per unit time in the alternating flow forming step P3.

As described above, according to the producing method for a cell structure body according to the embodiment of the disclosed technique, it is possible to produce a cell structure body having a structure suitable for culturing.

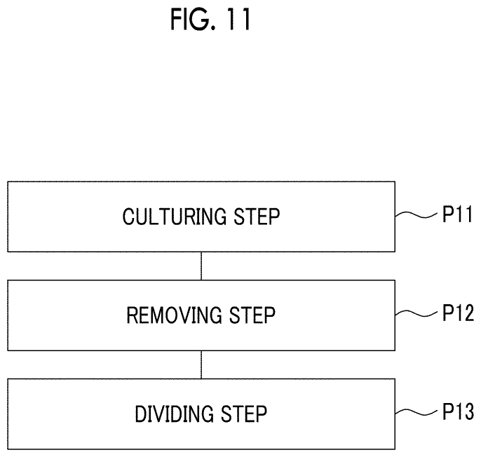

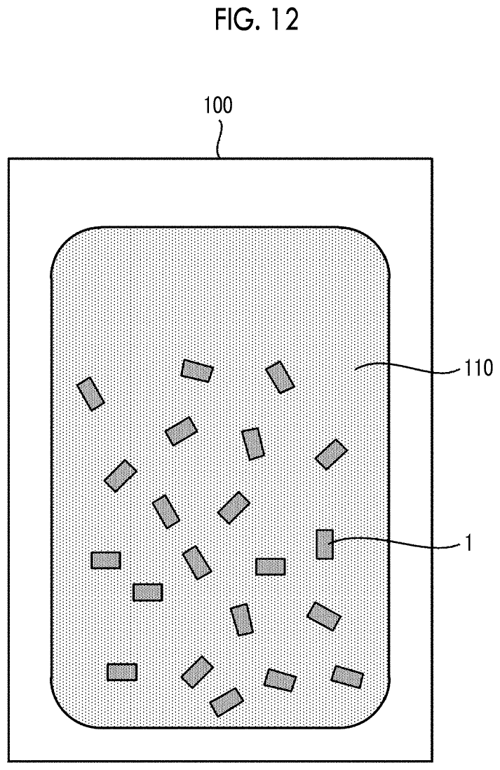

FIG. 11 is a step flow chart illustrating one example of a cell culturing method according to the embodiment of the disclosed technique. The cell culturing method according to the embodiment of the disclosed technique includes a culturing step P11 of culturing the cell structure body 1 produced according to the producing method described above, in a medium. For example, as illustrated in FIG. 12, a plurality of cell structure bodies 1 may be contained in a closed bag-shaped culture container 100 together with a medium 110 for culturing. The cell structure body 1 may be maintained to be in a suspended state in the medium 110 by adjusting the viscosity of the medium 110. Further, as necessary, the cell suspension containing the cell structure body 1 and the medium 110 may be cultured with stirring.

Oxygen and nutrients contained in the medium 110 can permeate the shell portion (the gel-like substance) of the cell structure body 1 and reach the core portion (the cells). In a case where the length L2 of the cell structure body 1 in the lateral direction is set to be 500 μm or less, the oxygen and the nutrients contained in the medium 110 can reach the center of the core portion. Further, in a case where the aspect ratio of the cell structure body 1 is set to be 100 or less, the risk of the cell structure bodies 1 being entangled with each other during the culture period can be suppressed, and the handling of the cell structure body 1 during the culture period is easy.

In a case where the cell structure body 1 is cultured, the cells in the core portion proliferate, and the size of the cell aggregate formed in the core portion increases. Since the entire circumference of the core portion is covered with the gel-like substance that constitutes the shell portion, cell proliferation does not occur indefinitely in the core portion, and the increase in the size of the cell aggregate formed in the core portion stops when the cell density in the core portion reaches a certain level. That is, the increase in the size of the cell aggregate formed in the core portion is limited by the shell portion covering the core portion.

As described above, according to the cell culturing method according to the present embodiment, in which the cell structure body 1 is used, the size of the cell aggregate does not expand beyond the size of the cell structure body 1, and thus it is possible to avoid an excessive increase in the size of the cell aggregate. Accordingly, the risk of the occurrence of cell death due to the insufficient supply of oxygen and nutrients to the central portion of the cell aggregate can be suppressed. That is, according to the cell culturing method according to the present embodiment, the cell survival rate can be increased, and thus the culture efficiency can be improved.

The cell culturing method according to the embodiment of the disclosed technique may further include a removing step P12 of removing the gel-like substance constituting the shell portion 11 after the culturing step P11 described above. The removal of the gel-like substance may be carried out, for example, after passing the predetermined period (for example, several days) after the start of the culturing step P11. As the predetermined period, for example, a period until the cells in the core portion reach a confluency of about 80% may be applied. The removal of the gel-like substance is carried out by, for example, dissociating the gel-like substance with a chelating agent such as EDTA.

As described above, since the outer shape of the cell structure body 1 according to the present embodiment is non-spherical and the aspect ratio is larger than 1, the gel volume fraction can be reduced as compared with that of the cell structure body 1X (see FIG. 2) according to Comparative Example 3 having a spherical outer shape, whereby the time for treating a chelating agent when removing the gel-like substance constituting the shell portion can be shortened.

The cell culturing method according to the embodiment of the disclosed technique may include a dividing step P13 of dividing the cell aggregate in the core portion exposed due to removing the gel-like substance constituting the shell portion into cell aggregates having a smaller size or single cells. The division of the cell aggregate may be carried out, for example, by passing the cell aggregate through a mesh. In addition, cell aggregate may be divided by treatment with a chemical solution that exhibits a cell dissociation effect. The divided cell aggregates can be reused for the production of the cell structure body 1. This enables the mass production of cells.

Table 1 below shows the evaluation results of the cell structure bodies produced by using the producing method according to the embodiment of the disclosed technique and the producing method according to Comparative Examples. The producing method, shape, and aspect ratio of the cell structure body, the contact angle of the gel precursor with respect to the inner wall of the micro flow path, the type of system (open system/closed system) in the producing method, the thickness of the shell portion in each of Examples and each of Comparative Examples are as shown in Table 1.

TABLE 1

| | Example 1 | Example 2 | Example 3 | Example 4 | Example 5 | Example 6 |
|---|---|---|---|---|---|---|
| Outline | vs | Typical | Shape | Shape | Small | Aspect ratio |
| | Comparative | example | changed | changed | contact | changed |
| | Example | | | | angle | |
| Producing | Individualized by gas injection | | | | | |
| method | | | | | | |
| Shape | Cylinder | Cylinder | Quadrangular | Hexagonal | Cylinder | Cylinder |
| | | | column | column | | |
| Aspect ratio | 50 | 10 | 10 | 10 | 10 | 100 |
| Contact angle | 100 | 100 | 100 | 100 | 60 | 100 |
| Open system/ | Closed | Closed | Closed | Closed | Closed | Closed |
| closed system | | | | | | |
| Average | 50 | 50 | 50 | 50 | 55 | 50 |
| thickness of | | | | | | |
| shell portion | | | | | | |
| Maximum | 60 | 60 | 60 | 60 | 70 | 60 |
| thickness of | | | | | | |
| shell portion | | | | | | |
| Minimum | 40 | 40 | 40 | 40 | 40 | 40 |
| thickness of | | | | | | |
| shell portion | | | | | | |
| Presence or | Absent | Absent | Absent | Absent | Absent | Absent |
| absence of | | | | | | |
| detects | | | | | | |
| Degree of | 3 | 3 | 3 | 3 | 2 | 3 |
| connection | | | | | | |
| Degree of | 2 | 3 | 3 | 3 | 3 | 2 |
| aggregation | | | | | | |
| EDTA | 5 | 4 | 4 | 4 | 4 | 5 |
| treatment time | | | | | | |
| | | | Comparative | Comparative | Comparative | Comparative |
| | Example 7 | Example 8 | Example 1 | Example 2 | Example 3 | Example 4 |
| Outline | Aspect ratio | Aspect ratio | Comparative Example | | | |
| | changed | changed | | | | |
| Producing | Individualized by | | Cut after fiber | Pulled after | Dropwise | Oxygen |
| method | gas injection | | formation | fiber | addition of liquid | plasma |
| | | | | formation | droplets to | treatment |
| | | | | | gelating agent | |
| Shape | Cylinder | Cylinder | Cylinder | Cylinder | Sphere | Cylinder |
| Aspect ratio | 3 | 1.5 | 50 | 50 | 1 | 10 |
| Contact angle | 100 | 100 | 100 | 100 | — | 30 |
| Open system/ | Closed | Closed | Closed | Closed | Open | Closed |
| closed system | | | | | | |
| Average | 50 | 50 | 50 | 60 | 50 | 60 |
| thickness of | | | | | | |
| shell portion | | | | | | |
| Maximum | 60 | 60 | 70 | 300 | 60 | 80 |
| thickness of | | | | | | |
| shell portion | | | | | | |
| Minimum | 40 | 40 | 0 | 40 | 40 | 40 |
| thickness of | | | | | | |
| shell portion | | | | | | |
| Presence or | Absent | Absent | Present | Absent | Absent | Absent |
| absence of | | | | | | |
| detects | | | | | | |
| Degree of | 3 | 3 | 3 | 2 | 3 | 1 |
| connection | | | | | | |
| Degree of | 3 | 3 | 2 | 2 | 3 | 2 |
| aggregation | | | | | | |
| EDTA | 3 | 2 | — | 4 | 1 | 4 |
| treatment time | | | | | | |

For Examples 1 to 8 and Comparative Examples 1, 2, and 4, a micro flow path having a flow path configuration described below was used. Specifically, a cylindrical glass capillary having an inner diameter of 0.6 mm and an outer diameter of 1.0 mm and an angular column having an inner diameter of 1 mm and an outer diameter of 1.5 mm were combined to prepare the micro flow path. One side of the cylindrical glass capillary was processed with a glass puller (PC-100, manufactured by NARISHIGE Group) so that the inner diameter was 0.25 mm.

The solution to be used was prepared as follows.

Cell suspension: Human iPS cells were seeded in Essential-8 medium (Thermo Fisher Scientific, Inc.) in a T-flask (Corning Inc.) coated with 0.5 μg/cm² of Vitronectin (Thermo Fisher Scientific, Inc.) so that the cell concentration was 2.0×10⁴ cells/cm² and were cultured for 4 days. The cultured human iPS cells were washed with DPBS (Thermo Fisher Scientific, Inc.), collected with TryPLE Select (Thermo Fisher Scientific, Inc.), centrifuged with a centrifuge (Thermo Fisher Scientific, Inc.) at 300 G for 4 minutes, and the cell suspension was adjusted in Essential-8 medium so that the cell concentration was 1.3×10⁷ cells/mL.

Gel precursor: 1.50 parts by mass of sodium alginate (FUJIFILM Wako Pure Chemical Corporation) and 0.85 parts by mass of sodium chloride (FUJIFILM Wako Pure Chemical Corporation) were dissolved in 97.65 parts by mass of distilled water. The resultant mixture was stirred until the mixture was completely dissolved and was subjected to sterilization by autoclaving at 120° C. for 60 minutes.

Gelating agent: 3 parts by mass of sucrose (FUJIFILM Wako Pure Chemical Corporation) and 1.11 parts by mass of calcium chloride were dissolved in 95.89 parts by mass of distilled water. The resultant mixture was stirred until the mixture was completely dissolved and was subjected to sterilization by autoclaving at 120° C. for 60 minutes.

Each of the prepared solutions was sent by a syringe pump (PD4000, manufactured by Harvard Apparatus) at the following flow rate.

Cell suspension: 50 μL/min

Gel precursor: 150 μt/min

Gelating agent: 4 mL/min

In addition, a mass flow meter (FCST1005FC-AIR, manufactured by Fujikin) was used for adjusting the flow rate of compressed air (original pressure: 0.4 MPa) as the gas, and the adjusted air was used for gas supply. All of the solutions and the gas were temperature-controlled indoors at 25° C. and used at the same temperature.

In Example 1, the aspect ratio of the cell structure body was set to be the same as that of Comparative Example 1 and Comparative Example 2 in consideration of the comparison with Comparative Example 1 and Comparative Example 2, and the aspect ratio of the cell structure body was set to be 50 by adjusting the flow rate of the gas to 120 μL/min and performing gas supply. In the cell structure body according to Example 1, the length L1 in the longitudinal direction was 11.5 mm, and the length L2 in the lateral direction was 0.23 mm. Example 2 is a typical example of the cell structure body according to the embodiment of the disclosed technique, and in Example 2, the aspect ratio of the cell structure body was set to be 10 by adjusting the flow rate of the gas to 450 μL/min and performing gas supply. In the cell structure body according to Example 2, the length L1 in the longitudinal direction was 2.3 mm, and the length L2 in the lateral direction was 0.23 mm.

In Examples 3 and 4, the shapes of the cell structure bodies were changed with respect to the typical example (Example 2) and were set to be respectively a quadrangular column and a hexagonal column. The shape of the cell structure body according to each of Examples 1, 2, and 5 to 9 and Comparative Examples 1 and 2 is cylindrical, and the shape of the cell structure body according to Comparative Example 3 is spherical. The shape of the cell structure body was changed by changing the shape of the cross section of the micro flow path used for producing the cell structure body.

In Example 5, the contact angle θ of the gel precursor with respect to the inner wall of the micro flow path was set to be smaller than that of the typical example (Example 2). In other words, Examples 1 to 4 and 6 to 8 and Comparative Examples 1 and 2 were produced using a micro flow path which was subjected to a water repellency treatment on the inner wall surface, and Example 5 was produced using a micro flow path (a glass capillary) which was not subjected to a water repellency treatment. The water repellency treatment in each of Examples 1 to 4 and 6 to 8 and Comparative Examples 1 and 2 was carried out according to the following procedure. A solution in which the ratio of 1% by mass acetic acid to ethanol was 20:80 was prepared, a silane coupling agent KBE-3083 manufactured by Shin-Etsu Chemical Co., Ltd. was added to the prepared solution to a concentration of 1% by mass, and hydrolysis was carried out with stirring for 1 hour at room temperature. A glass micro flow path was immersed in this solution for 6 hours, dried at room temperature, and then heated for 10 minutes on a hot plate, a surface temperature of which was set to 100° C. Comparative Example 4 was produced using a micro flow path composed of a glass capillary treated with an oxygen plasma processing machine (PR-500, manufactured by Yamato Scientific Co., Ltd.) at 15 Pa for 1 minute.

The contact angle of the gel precursor with respect to the inner wall of the micro flow path was acquired by the following procedure. 100 μl of the dissociable hydrogel precursor solution was injected into the main flow path of the micro flow path using a micropipette, observed under an inverted microscope (AXIO Observer Z1, manufactured by Carl Zeiss AG), and a transmission image of the gas-liquid interface was photographed. From this image, as illustrated in FIG. 7, the angle which is formed by the interface of the dissociable hydrogel precursor solution and the air and the inner wall surface of the main flow path was acquired as the contact angle θ. In a case where the water repellency treatment was applied (Examples 1 to 4 and 6 to 8 and Comparative Examples 1 and 2), the contact angles θ were 100°, respectively. On the other hand, the contact angle θ of Example 5 to which the water repellency treatment was not applied was 60°. The contact angle θ of Comparative Example 4 in which a glass capillary subjected to an oxygen plasma treatment was used was 30°.

In Examples 6, 7, and 8, the respective aspect ratios of the cell structure bodies were changed with respect to the typical example (Example 2). In Example 6, the aspect ratio of the cell structure body was set to be 100 by adjusting the flow rate of the gas to 70 μL/min and performing gas supply. In the cell structure body according to Example 6, the length L1 in the longitudinal direction was 23.0 mm, and the length L2 in the lateral direction was 0.23 mm. In Example 7, the aspect ratio of the cell structure body was set to be 3 by adjusting the flow rate of the gas to 3.1 mL/min and performing gas supply. In the cell structure body according to Example 7, the length L1 in the longitudinal direction was 0.69 mm, and the length L2 in the lateral direction was 0.23 mm. In Example 8, the aspect ratio of the cell structure body is set to 1.5 by adjusting the flow rate of the gas to 5.2 mL/min and performing gas supply. In the cell structure body according to Example 8, the length L1 in the longitudinal direction was 0.35 mm, and the length L2 in the lateral direction was 0.23 mm.

In Comparative Example 1, a fibrous cell structure body was formed using a micro flow path, and then the fibrous cell structure body was individualized by cutting with a cutter. In Comparative Example 2, a fibrous cell structure body was formed using a micro flow path, and then the fibrous cell structure body was individualized by tearing off. Comparative Example 3 was produced by dropwise adding liquid droplets containing cells and a gel precursor to a gelating agent. That is, Comparative Example 3 was produced without using a micro flow path, and the producing environment of the cell structure body is an open system. The shape of the cell structure body according to Comparative Example 3 is spherical due to the producing method thereof, and thus the aspect ratio of the cell structure body is 1. In Comparative Example 4, as described above, the contact angle θ was set to be 30° by using the glass capillary subjected to an oxygen plasma treatment. All of the cell structure bodies according to Examples 1 to 8 and Comparative Examples 1 to 4 are cell structure bodies of a core-shell type cell structure body, containing a core portion consisting of a cell suspension containing iPS cells and a shell portion consisting of a gel-like substance covering the circumference of the core portion.

Regarding the cell structure body according to Examples 1 to 8 and Comparative Examples 1 to 4, the presence or absence of defects in the shell portion was confirmed. Regarding the cell structure body according to Comparative Example 1, the core portion was exposed at the cut surface cut by a cutter, and the cell suspension of the core portion leaked from the cut surface. That is, the cell structure body according to Comparative Example 1 was a structure having a defect in the shell portion. Regarding the cell structure bodies according to Examples 1 to 8 and Comparative Examples 2 to 4, no defects were confirmed in the shell portion.

One hundred cell structure bodies according to each of Examples 1 to 8 and Comparative Examples 1 to 4 were observed under an inverted microscope (AXIO Observer Z1, manufactured by Carl Zeiss AG), and the degree of connection, which indicates the number proportion of the cell structure bodies in which a shell portion of one cell structure body is connected to a shell portion of another cell structure body, was evaluated according to the following criteria. It has been confirmed that the degree of connection is high in the cell structure bodies according to Example 5 and Comparative Example 4, in which the contact angle of the gel precursor with respect to the inner wall of the micro flow path is small.

1: The degree of connection is 25% or more.

2: The degree of connection is 1% or more and less than 25%.

3: The degree of connection is 0%.

A cell structure body dispersion was prepared from the cell structure body according to each of Examples 1 to 8 and Comparative Examples 1 to 4 so that the cell density thereof was 1.3×10⁵ cells/mL. The cell structure body dispersion was prepared by precipitating and discarding the supernatant and diluting with Essential 8 medium (manufactured by Thermo Fisher Scientific, Inc.). The cell density in the cell structure body dispersion was calculated from the cell density of the cell suspension in the core portion and the amount of each solution sent at the time of producing the cell structure body. 20 mL of the cell structure body dispersion according to each Example and each Comparative Example was gently stirred with a stirrer (having a length of 1 mm, made of PTFE) at 100 rpm for 5 minutes and then allowed to stand for 10 minutes. One hundred cell structure bodies according to each Example and Comparative Example, which were precipitated by this operation, were observed under an inverted microscope (AXIO Observer Z1, manufactured by Carl Zeiss AG), and the degree of aggregation, which indicates the number proportion of the cell structure bodies in which cell structure bodies are entangled with each other, was evaluated according to the following criteria. It has been confirmed that the degree of aggregation is high in the cell structure bodies according to Examples 1 and 6 and Comparative Examples 1 and 2, in which the aspect ratio of the cell structure body is high.

1: The degree of aggregation is 25% or more.

2: The degree of aggregation is 1% or more and less than 25%.

3: The degree of aggregation is 0%.

The cell structure body according to each of Examples 1 to 8 and Comparative Examples 1 to 4 was cultured for 4 days, washed with Phosphate Buffered Saline (PBS), and then immersed in 0.5 mmol/1-EDTA (manufactured by Nacalai Tesque, Inc.). The cell structure body under the EDTA treatment was observed under a phase-contrast microscope, and the time during which the gel-like substance in the shell portion was completely degraded was evaluated according to the following criteria. It has been confirmed that the lower the aspect ratio of the cell structure body is, the longer the EDTA treatment time is.

1: 3 minutes or more

2: 1 minute or more and less than 3 minutes

3: 30 seconds or more and less than 1 minute

4: 5 seconds or more and less than 30 seconds

5: Less than 5 seconds

A sample of the cell structure body corresponding to Example 2, which was prepared so that the cell density of hiPS cells in the core portion was 1.3×10⁷ cells/mL, was cultured in Essential 8 medium (manufactured by Thermo Fisher Scientific, Inc.) for 4 days while exchanging the medium every 24 hours. The sample on the 4th day of culture was observed under a phase-contrast microscope. It has been confirmed by trypan blue staining that the hiPS cells in the core portion proliferate while being alive.

The above sample cultured for 4 days was washed with PBS and then immersed in 0.5 mmol/1-EDTA (manufactured by Nacalai Tesque, Inc.) for 1 minute. The sample under the EDTA treatment was observed under a phase-contrast microscope. It has been confirmed that in the aggregate of the hiPS cells of the core portion, the gel-like substance forming the shell portion is lysed while the shape of the core portion is maintained.

What is claimed is:

1. A producing method for a cell structure body in which a circumference of a cell suspension containing a plurality of cells is covered with a gel-like substance, the producing method comprising:
causing a cell suspension to flow into a tubular flow path;
injecting a gel precursor into the flow path to cover the circumference of the cell suspension with the gel precursor;
injecting a gas into the flow path to form, in the flow path, an alternating flow obtained from the gas and the cell suspension covered with the gel precursor; and
injecting a gelating agent into the flow path and causing the gelating agent to join the alternating flow to gelate the gel precursor.

2. The producing method according to claim 1,
wherein an injection amount of the gas that is injected into the flow path per unit time is constant.

3. The producing method according to claim 1,
wherein an aspect ratio of the cell structure body is controlled by an injection amount of the gas that is injected into the flow path per unit time.

4. The producing method according to claim 1,
wherein the gas is intermittently injected into the flow path.

5. The producing method according to claim 1,
wherein a cross section of the flow path, which is orthogonal to a liquid passage direction, has a circular or polygonal shape.

6. The producing method according to claim 1,
wherein a contact angle of the gel precursor with respect to an inner wall surface of the flow path is 50° or more.

7. The producing method according to claim 6,
wherein the inner wall surface of the flow path has been subjected to a water repellency treatment.

8. A cell culturing method for culturing cells contained in a cell structure body produced by the producing method according to claim 1, the cell culturing method comprising:
culturing the cell structure body in a medium.

9. The cell culturing method according to claim 8, further comprising:
removing the gel-like substance after the culturing.

10. A cell structure body comprising:
a cell suspension containing a plurality of cells; and
a gel-like substance covering an entire circumference of the cell suspension,
wherein over an entire region, a thickness of the gel-like substance is 0.1 times to 3 times an average thickness of the gel-like substance, and
the cell structure body has a non-spherical outer shape and an aspect ratio of 100 or less.

11. The cell structure body according to claim 10,
wherein the aspect ratio is 20 or less.

12. The cell structure body according to claim 10,
wherein a length in a lateral direction is 10 μm or more and 500 μm or less.

13. A micro flow path comprising:
a tubular main flow path through which a cell suspension flows;
a first branch flow path that communicates with a first injection port into which a gel precursor is injected, and joins the main flow path at a first joining portion;
a second branch flow path that communicates with a second injection port into which a gas is injected, and joins the main flow path at a second joining portion of the main flow path, the second joining portion being disposed downstream of the first joining portion in a liquid passage direction; and
a third branch flow path that communicates with a third injection port into which a gelating agent gelating the gel precursor is injected, and joins the main flow path at a third joining portion of the main flow path, the third joining portion being disposed downstream of the second joining portion in the liquid passage direction.
